(19) United States
(12) Patent Application Publication
(10) Pub. No.: US 2023/0026261 A1
(43) Pub. Date: Jan. 26, 2023

(54) NANOHOLE ARRAY SENSOR ELEMENTS AND SENSORS INCLUDING THE SAME

(71) Applicant: 3M INNOVATIVE PROPERTIES COMPANY, St. Paul, MN (US)

(72) Inventors: Myungchan Kang, Woodbury, MN (US); Daniel M. Lentz, Cottage Grove, MN (US); Masayuki Nakamura, Woodbury, MN (US); Karl K. Stensvad, Eagan, MN (US); James Zhu, Woodbury, MN (US)

(21) Appl. No.: 17/756,392

(22) PCT Filed: Dec. 11, 2020
(86) PCT No.: PCT/IB2020/061821

§ 371 Date: May 24, 2022

Related U.S. Application Data
(60) Provisional application No. 62/951,535, filed on Dec. 20, 2019.

(51) Int. Cl. G01N 21/552

(52) U.S. Cl. CPC G01N 21/554

(57) ABSTRACT

A sensor element and sensor comprising the sensing element. The sensor includes, in order, an absorptive layer, a metallic layer and an optically transparent, dielectric substrate. The absorptive layer has a polymer of intrinsic microporosity having an average pore volume of at least 0.1 nm³. The metallic layer has a plurality of openings each extending from the first to the second major surface of the metallic layer, the openings having a pitch in a range from 50 nm to 5000 nm, wherein the openings have an opening size in a range from 5% to 95% percent of the pitch.

### FIELD OF INVENTION

The present invention relates broadly to sensors, and more particularly to nanohole array sensors based on surface plasmon resonance. The sensors can be used, for example, to quantitatively detect volatile organic compounds.

### BACKGROUND

The ability to detect chemical vapors, especially volatile organic compounds (VOCs), is important in a number of areas, including health and environmental safety. Such detection and/or monitoring of organic vapors may find particular use in, for example, so called “end of service life indicators” which are desired for personal protective equipment, such as respirators.

Many methods for the detection of chemical analytes have been developed, including optical, gravimetric, and microelectromechanical (MEMS) methods. In particular, sensors that monitor electrical properties such as capacitance, impedance, resistance, etc., have been developed. Often such sensors rely on the change that occurs in the electrical properties of a material upon adsorption of an analyte onto, or absorption of an analyte into, the material.

Surface plasmon resonance spectroscopy has received recent attention as an analytical technique for detection of organic vapors. Surface plasmon resonance spectroscopy is an optical sensing technique in which surface plasmons are created at the boundary between a metal surface and dielectric. Surface plasmons are surface electromagnetic waves that propagate in a direction parallel to a metal/dielectric (or metal/vacuum) interface. Since the wave is on the boundary of the metal and the dielectric, these oscillations are very sensitive to any change in the environment at the boundary, such as the adsorption of molecules to the metal surface.

Surface plasmon resonance spectroscopy typically utilizes a thin metallic layer disposed onto an optically transparent substrate (e.g., glass). Electromagnetic radiation (e.g., ultraviolet light, visible light or infrared light) is directed at the thin metallic layer through the transparent substrate to create an evanescent wave at the surface of the thin metallic layer opposite the source of electromagnetic radiation. In one variation, termed the Kretschmann configuration, the detector analyzes the light reflected off the surface of the metal. In an alternative variation, the metallic layer comprises a periodic array of nanoholes (i.e., nanohole array) having subwavelength dimensions, and the detector analyzes the electromagnetic radiation transmitted through the metallic layer on the side opposite the source of electromagnetic radiation. The latter configuration provides for a simpler sensor having a compact, collinear optical arrangement with higher analyte sensitivity.

### SUMMARY

The present disclosure describes unique nanohole array sensing elements for a plasmon resonance sensor that can be used to quantitatively detect and measure VOCs based upon the refraction of transmitted light.

In one embodiment, the present disclosure provides a sensor element comprising, in order, an absorptive layer having first and second major surfaces, a metallic layer disposed on the second major surface of the absorptive layer, and an optically transparent, dielectric substrate having first and second opposed major surfaces with the metallic layer disposed on the first major surface of the substrate. The absorptive layer comprises a polymer of intrinsic microporosity having an average pore volume of at least 0.1 nm³. The metallic layer has first and second opposed major surfaces and a plurality of openings each extending from the first to the second major surface of the metallic layer, the openings having a pitch in a range from 50 nm to 5000 nm, wherein the openings have an opening size in a range from 5% to 95% percent of the pitch.

In another embodiment, the present disclosure provides a sensor for organic solvents comprising the sensor elements of the present disclosure.

In a further embodiment, the present disclosure provides a sensor comprising a light source, a sensor element of the present disclosure, and a detector that measures optical transmission through the sensor element.

Sensors comprising the nanohole array sensor elements of the present disclosure are configured to measure the electromagnetic radiation transmitted by the sensing element. In contrast to devices utilizing the Kretschmann configuration, the sensors in the present disclosure are simpler, more compact, able to use single and multiple wavelength sources, and exhibit greater sensitivity to target analytes (e.g., VOCs).

As used herein:

The term “comprises” and variations thereof do not have a limiting meaning where these terms appear in the description and claims. Such terms will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

In this application, terms such as “a,” “an,” and “the” are not intended to refer to only a singular entity, but include the general class of which a specific example may be used for illustration. The terms “a,” “an,” and “the” are used interchangeably with the phrases “at least one” and “one or more.” The phrases “at least one of” and “comprises at least one of” followed by a list refers to any one of the items in the list and any combination of two or more items in the list.

The term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and in certain embodiments, by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Herein, “up to” a number (e.g., up to 50) includes the number (e.g., 50).

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

Reference throughout this specification to “some embodiments” means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The term “light” generally refers to electromagnetic radiation in the ultraviolet, visible and infrared regions of the electromagnetic spectrum.

The above summary of the present disclosure is not intended to describe each disclosed embodiment or every implementation of the present disclosure. The description that follows more particularly exemplifies illustrative embodiments.

### BRIEF DESCRIPTION OF THE DRAWINGS

FIG. 1 is a schematic top plan view of one embodiment of a sensor element in the present disclosure;

FIG. 2A is a schematic cross-sectional view of the sensor element along line 2-2 in FIG. 1;

FIG. 2B is a schematic cross-sectional view of the sensor element along line 2-2 in FIG. 1 with the absorbent layer removed;

FIG. 3 is a schematic side view of one embodiment of a sensor in the present disclosure;

FIG. 4 is an SEM image of a metallic layer with a periodic array of nanoholes that can be used in sensor elements in the present application;

FIG. 5A is the absorbance spectra for Example 1 using a sensing element without PIM;

FIG. 5B is the plot of peak position vs. time based upon the absorbance spectra in FIG. 5A;

FIG. 6A is the absorbance spectra for Comparative Example 1 using a sensing element with PIM;

FIG. 6B is a plot of peak position vs. time based upon the absorbance spectra in FIG. 6A.

Unless otherwise indicated, all figures and drawings in this document are not to scale and are chosen for the purpose of illustrating different embodiments of the invention. In particular, the dimensions of the various components are depicted in illustrative terms only, and no relationship between the dimensions of the various components should be inferred from the drawings, unless so indicated.

### DETAILED DESCRIPTION

In the following description of illustrative embodiments, reference is made to the accompanying figures of the drawing which form a part hereof, and in which are shown, by way of illustration, specific embodiments. It is to be understood that other embodiments may be utilized and structural changes may be made without departing from the scope of the present invention.

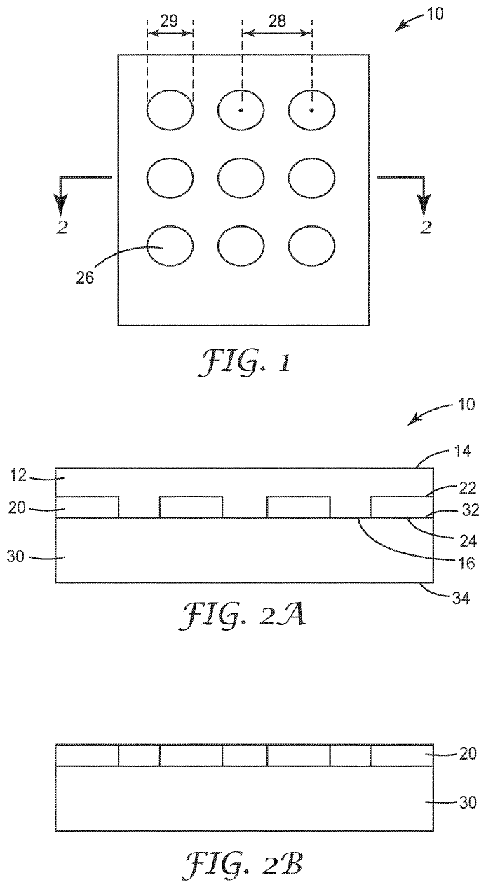

FIGS. 1 and 2 illustrate one embodiment of a sensor element 10 comprising, in order, an absorptive layer 12 having a first major surface 14 and a second major surface 16, a metallic layer 20 disposed on the second major surface 16 of the absorptive layer 12, and an optically transparent, dielectric substrate 30 having a first major surface 32 and a second opposed major surfaces 34 with the metallic layer 20 disposed on the first major surface 32 of the substrate.

The absorptive layer 12 comprises a polymer of intrinsic microporosity (PIM). The term “microporosity” means that the polymer has a significant amount of internal, interconnected pore volume, with the mean pore size (as characterized, for example, by sorption isotherm procedures) being less than about 100 nanometers (nm), typically less than about 10 nm. Such microporosity provides that molecules of an organic analyte will be able to penetrate the internal pore volume of the polymer.

PIMs of the present application typically have an average pore volume of at least 0.1 nm³. In some embodiments, the PIMs have an average pore volume of at least 0.2, 0.25, 0.3, 0.4, 0.5, 0.6, 0.7, 0.75, 0.8, 0.9, 1, 1.25, 1.5, 2, or 2.5 nm³. Pore volume may be determined by positron annihilation lifetime spectroscopy (PALS), for example, as described by de Miranda et al. in Physical Status Solidi RRL, 2007, vol. 1, No. 5, pp. 190-192.

In general, PIMs can be formulated via the use of any combination of monomers that form a very rigid polymer within which there are sufficient structural features to induce a contorted structure. In various embodiments, PIMs can comprise organic macromolecules comprised of generally planar species connected by rigid linkers, said rigid linkers having a point of contortion such that two adjacent planar species connected by the rigid linkers are held in non-coplanar orientation. In further embodiments, such materials can comprise organic macromolecules comprised of first generally planar species connected by rigid linkers predominantly to a maximum of two other said first species, said rigid linkers having a point of contortion such that two adjacent first planar species connected by the rigid linkers are held in non-coplanar orientation. In various embodiments, such a point of contortion may comprise a spiro group, a bridged ring moiety or a sterically congested single covalent bond around which there is restricted rotation.

In a polymer with such a rigid and contorted structure, the polymer chains are unable to pack together efficiently, thus the polymer possesses intrinsic microporosity. Thus, PIMs have the advantage of possessing microporosity that is not significantly dependent on the thermal history of the material. PIMs thus may offer advantages in terms of being reproducibly manufacturable in large quantities, and in terms of not exhibiting properties that change upon aging, shelf life, etc.

Exemplary PIMs can be prepared by step-growth polymerization where at least one bis-catechol (A) is allowed to react with at least one fluorinated arene (B) under basic conditions as shown in Scheme I (below) according to the procedure reported by Budd et al. in Chemical Communications, 2004, (2), pp. 230-231.

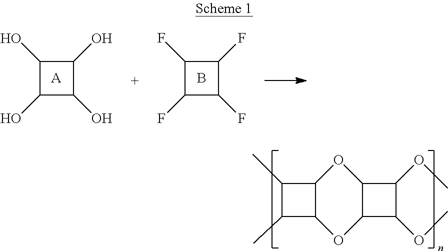

Due to the rigidity and contorted nature of the backbone of the resulting polymers, these polymers are unable to pack tightly in the solid state and thus have at least 10 percent free volume and are intrinsically microporous.

In some embodiments, the PIM may be a homopolymer having a monomeric unit selected from the group consisting of

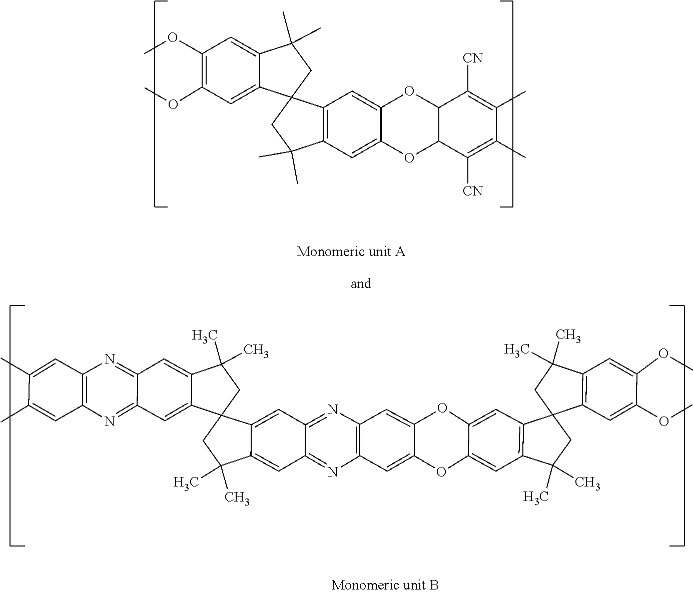

which homopolymers respectively correspond to PIM-1 and PIM-7 in de Miranda et al., Physical Status Solidi RRL, 2007, vol. 1, No. 5, pp. 190-192, which reports that PIM-1 and PIM-7 at room temperature have an average radius (i.e., average pore radius) of 0.48 nm and an average volume (i.e., average pore volume) of 0.47 nm³ as determined by positron annihilation lifetime spectroscopy (PALS).

PIM homopolymers having monomeric unit A can be formed by step-growth polymerization as described by Budd et al. in Advanced Materials, 2004, vol. 16, No. 5, pp. 456-459. PIM homopolymers having monomeric unit B can be formed according to the method described by Budd et al. in Journal of Membrane Science, 2005, vol. 251, pp. 263-269.

In one embodiment, the PIM is a hydrophobic material (e.g., a hydrophobic organic polymeric material), that will not absorb liquid water to an extent that the material swells significantly or otherwise exhibits a significant change in a physical property. Such hydrophobic properties are useful in providing an organic analyte sensor element that is relatively insensitive to the presence of water. The material may however comprise relatively polar moieties for specific purposes.

In one embodiment, the PIM comprises a continuous matrix. Such a matrix is defined as an assembly (e.g., a coating, layer, etc.) in which the solid portion of the material is continuously interconnected (irrespective of the presence of porosity as described above, or of the presence of optional additives as discussed below). That is, a continuous matrix is distinguishable from an assembly that comprises an aggregation of particles (e.g., zeolites, activated carbons, carbon nanotubes, etc.). For example, a layer or coating deposited from a solution will typically comprise a continuous matrix (even if the coating itself is applied in a patterned manner and/or comprises particulate additives). A collection of particles deposited via powder spraying, coating and drying of a dispersion (e.g., a latex), or by coating and drying of a sol-gel mixture, may not comprise a continuous network as defined by applicant. However, if such a latex, sol-gel, etc., layer can be consolidated such that individual particles are no longer discernible, nor is it possible to discern areas of the assembly that were obtained from different particles, such a layer may then meet applicant's definition of a continuous matrix.

The absorptive layer comprising the PIM may be deposited by any suitable technique, including solvent coating, spin coating, dip coating, transfer coating and screen printing. The coatings are typically air dried to remove the solvent and/or may be heated, for example, to a temperature in a range of from 100° C. to 200° C. to further dry the coated PIM. The absorptive layer may comprise one or more additional components such as, for example, antioxidants, fillers, residual solvent, wetting aids, leveling agents. The average thickness of the absorptive layer is typically in a range of from 10 nm to 10,000 nm. In some embodiments, the average thickness is in the range from 100 nm to 3,000 nm, 200 nm to 1,000 nm, or even 300 nm to 800 nm.

The metallic layer 20 has a first major surface 22 and second opposed major surface 24. The metallic layer 20 comprises at least one of gold, silver, aluminum, copper, platinum, ruthenium, nickel, palladium, rhodium, iridium, chromium, iron, lead, tin, zinc, a combination thereof (e.g., layers of, or co-deposited, platinum and ruthenium), and an alloy thereof (e.g., Pt—Fe alloys).

The metallic layer 20 has a plurality of openings 26 each extending from the first major surface 22 to the second major surface 24 of the metallic layer 20. The openings 26 can be one of circular, oval, or polygonal (e.g., triangular, square, or rectangular). The openings typically form a repeating pattern, such as a square lattice, a rectangular lattice, a hexagonal lattice, a rhombic lattice, or a parallelogrammic lattice.

The openings 26 typically have a pitch 28 in a range from 50 to 5000 nm. As used herein, the term “pitch” refers to the distance from the center of one opening to the center of the next nearest opening. In some embodiments, the openings have a pitch in a range from 100 to 2500, 100 to 1000, 250 to 100, or even 300 to 900 nm. The openings 26 typically have an opening size 29 in a range from 5 to 95 percent of the pitch. In some embodiments, the opening size is in a range from 10 to 90, 15 to 85, or even 20 to 80 percent of the pitch. The opening size of a circular opening is the diameter of the opening. The opening size for an oval opening is the length of its major axis. The opening size for a polygonal opening is based upon the length of the longest line that can be drawn from one vertex, through the center of the opening, to the opposite side of the opening.

Typically, the metallic layer has an average thickness in a range from 5 nm to 1000 nm. In some embodiments, the average thickness is in a range from 25 nm to 500 nm, 25 nm to 250 nm, or even 50 nm to 200 nm.

The metallic layer may be deposited according to any suitable technique, including, for example, thermal vapor deposition, sputtering techniques, electrodeposition, and electroless plating.

The dielectric substrate 30 comprises at least one of an optically transparent inorganic layer or an optically transparent polymeric layer. Exemplary inorganic layers comprise at least one of glass, SiN and SiO₂. Exemplary polymeric layers comprise polyethylene terephthalate, poly(methyl methacrylate), polyvinyl chloride, polyethylene, polypropylene, styrene methyl methacrylate, polycarbonate, polystryrene, and copolymers thereof.

The sensor element can be produced in the form of a roll and cut to size as needed. Alternatively, the sensor element can be manufactured in sheets. The sensor element can also be framed to produce an article for ease of handling.

In some embodiments, the sensor element comprises an optically transparent, dielectric supporting layer adjacent the dielectric substrate 30. An exemplary support layer comprises a glass made of fused silica.

FIG. 3 illustrates one embodiment of a sensor comprising a sensor element of the present disclosure. The sensor 40 comprises a chamber 42, an inlet port 44, and outlet port 46, a first optical window 48 and a second optical window 50. The sensor 40 further comprises a light source 52 proximate the first optical window 48 and a detector 54 proximate the second optical window 50, where the light source 52 and detector 54 are in optical alignment.

The light source can be a single wavelength light source, such as a single wavelength light emitting diode (LED) or laser. Alternatively, the light source can be a multiple wavelengths light source, such as a white light source.

In some embodiments, the light source emits at least one wavelength of ultraviolet (UV) light (i.e., 10 to 400 nanometers). In other embodiment, the light source emits at least one wavelength of visible light (i.e., greater than 400 to less than 700 nanometers). In yet other embodiment, the light source emits at least one wavelength of infrared (IR) light (i.e., 700 to 1,000,000 nanometer).

The detector senses the optical transmission through the sensor element. Depending on the amount of analyte vapor absorbed by the absorptive layer, the dielectric constant of the absorptive layer will change, resulting in the change of distinctive spectral features. The distinctive spectral features include peak/valley positions, measured intensity at specified wavelengths, some mathematic processes using several intensities at various wavelengths (e.g. relative intensity ratio at two wavelengths), Hue, and so on. The detector response can be correlated to the concentration of analyte vapor present in the vapor delivery chamber.

The detector comprises at least one photodetector, such as a photodiode, a monochromator, a photoresistor, a phototransistor, a charge-coupled device, a complementary metal-oxide-semiconductor image sensor, a photomultiplier tube, and a phototube.

The sensor can be used to measure the presence of organic solvents. Typically, the solvent is a volatile organic compound; however, this is not a requirement. Examples of suitable analyte vapors include aliphatic hydrocarbons (e.g., n-octane or cyclohexane), ketones (e.g., acetone or methyl ethyl ketone), aromatic hydrocarbons (benzene, toluene, chlorobenzene, or naphthalene), nitriles (e.g., acetonitrile or benzonitrile), chlorinated aliphatic hydrocarbons (e.g., chloroform, dichloroethane, methylene chloride, carbon tetrachloride, or tetrachloroethylene), esters (e.g., vinyl acetate, ethyl acetate, butyl acetate, or methyl benzoate), sulfides (e.g., phenyl mercaptan), ethers (e.g., methyl isobutyl ether or diethyl ether, aldehydes (e.g., formaldehyde, benzaldehyde, or acetaldehyde), alcohols (e.g., methanol or ethanol), amines (e.g., 2-aminopyridine), organic acids (e.g., acetic acid, propanoic acid), isocyanates (e.g., methyl isocyanate or toluene-2,4-diisocyanate), and nitro-substituted organics (e.g., nitromethane or nitrobenzene).

In operation, the sensor element 10 is placed in the chamber 42 with the substrate 30 facing the first optical window 48 and the absorbent material facing the second optical window 50. A carrier gas containing the organic analyte flows into the chamber 42 through inlet port 44 and out of the chamber through outlet port 46. A light source is directed onto the sensor element 10 through first optical window 48. Detector 54 measures the optical transmission through the sensor element 10.

### EXAMPLES

Objects and advantages of this invention are further illustrated by the following examples, but the particular materials and amounts thereof recited in these examples, as well as other conditions and details, should not be construed to unduly limit this invention. These examples are merely for illustrative purposes only and are not meant to be limiting on the scope of the appended claims.

The following abbreviations are used in this section: L=liters, mL=milliliters, g=grams, mol=mole, cm=centimeters, mm=millimeters, μm=micrometers, nm=nanometers, wt=weight, sec=seconds, min=minutes, h=hours, ppm=parts per million, ° C.=degrees Celsius, K=Kelvin, cP=centipoise, mM=millimolar, Pa=pascals, mA=milliamperes. Abbreviations for materials used in this section, as well as descriptions of the materials, are provided in Table 1.

TABLE 1
Materials

| Material | Details |
|---|---|
| MEK | Methyl Ethyl Ketone, J. T. Baker, Center Valley, PA |
| PET | Heat stabilized polyester film, available under |
| | the trade designation MELINEX ST504, Tekra, New |
| | Berlin, WI |
| PIM | Polymer of intrinsic microporosity, prepared as |
| | described below in “Preparation of PIM” |
| PDMS | Vinyl-terminated polydimethylsiloxane, available |
| | under the trade designation SYLGARD 184 from |
| | Sigma-Aldrich |
| octadecanethiol | 1-octadecanethiol, available from Sigma-Aldrich |

### Preparation of PIM Solution

4.0˜4.4% (wt/wt) PIM solid dissolved in chlorobenzene was used. To prepare PIM solid, 130 g of 5,5′,6,6′-tetrahydroxy-3,3,3′,3′-tetramethyl-1,1′-spirobisindane (Part number B22170 from Alfa Aesar, Tewksbury, Mass.) were combined with 77.1 g of tetrafluoroterephthalonitrile (Part number H61326 from Alfa Aesar), 322.83 g potassium carbonate, and 3380 g of N,N-dimethylformamide, and the mixture was reacted at 68° C. for 72 h. The polymerization mixture was poured into water, and the precipitate was isolated by filtration. The resulting polymer was twice dissolved in tetrahydrofuran, precipitated from methanol, and air dried at room temperature. A yellow solid product obtained was dissolved in chlorobenzene and 4.0˜4.4% (wt/wt) PIM solution was prepared. The viscosity of solution was 3.0˜7.0 cP using a Rheometer.

### Preparation of Nanohole Array

Nanohole arrays were produced in gold layers coated on PET substrates via a microcontact printing process. PDMS was cast against an e-beam written master containing the target pattern. The resulting PDMS stamp was then inked in 6.5 mM octadecanethiol dissolved in ethanol for 16 h. Following this inking step, the stamp was blown off with nitrogen and allowed to dry overnight. The stamp was then mounted in a microcontact printing device. PET with 100 nm (nominal thickness) evaporated gold was loaded onto the drum of the printer, and the stamp was contacted to the film in order to transfer a self-assembled monolayer of the octadecanethiol. Finally, this octadecanethiol was used as an etch resist in an immersed rotational wet etch using an aqueous solution 20 mM in ferric nitrate and 30 mM in thiourea for approximately 25 min. The samples were rinsed thoroughly with DI water prior to subsequent characterization and testing.

### Characterization

### Scanning Electron Microscopy (SEM)

SEM images were obtained using a SEM obtained under the trade designation JCM-5000 NEOSCOPE from JEOL USA, Peabody, Mass. A JEOL aluminum specimen mount (25 mm dia., 10 mm thick, Ted Pella, #16153) was used for mounting samples in the SEM. A small piece of conductive carbon tape, obtained under the trade designation 3M XYZ/ISOTROPIC ELECTRICALLY CONDUCTIVE ADHESIVE 9712 from 3M Company, St. Paul, Minn., was placed directly on the surface of the mount, and samples were mounted by affixing a small piece of the sample onto the carbon tape. A small amount of conductive silver paint, obtained under the trade designation PELCO COLLOIDAL SILVER, part number 16034, from TED PELLA, Redding, Calif., was then applied to a small region of each sample piece, and extended to contact either the carbon tape, aluminum mount surface or both. After briefly allowing the paint to air dry at room temperature, the mounted sample assembly was placed into a sample preparation system, obtained under the trade designation DESK V from DENTON VACUUM, Moorestown, N.J., and the chamber evacuated to ˜0.04 Torr (5.3 Pa). Argon gas was then introduced into the sputtering chamber until the pressure stabilized at ˜0.06 Torr (8.0 Pa) before initiating the plasma and sputter coating gold onto the assembly for 120 see at ˜30 mA.

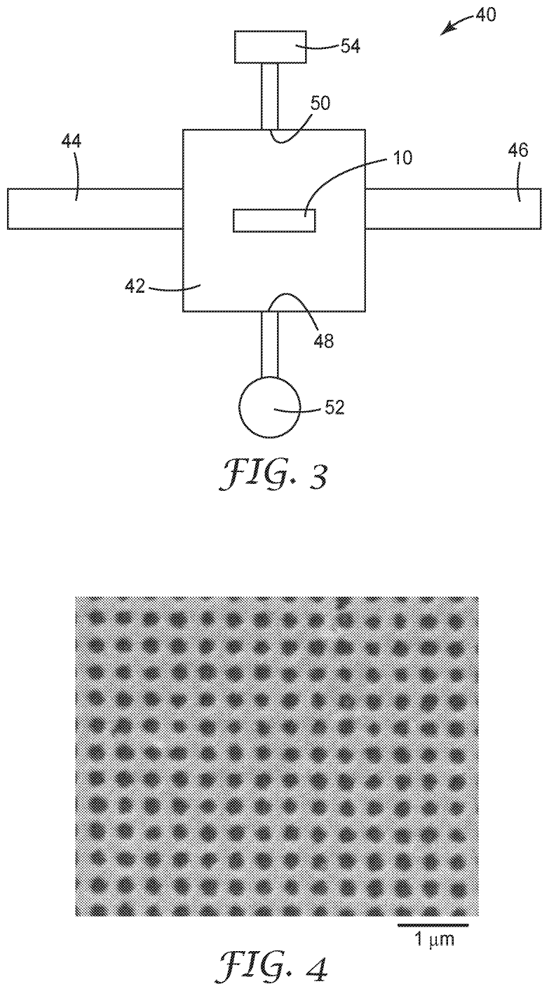

FIG. 4 shows a SEM image of nanohole array. Using a computer program Image J (software available from National Institutes of Health, v 1.37), the SEM image was analyzed. The SEM image was converted into a binary image using a threshold point. The number of holes and hole area were calculated from black pixels. The average hole diameter was 237 nm. The average center to center pitch between holes was 400 nm.

### Atomic Force Microscopy (AFM)

AFM studies were performed with an AFM obtained under the trade designation MOBILE S from Nanosurf, Woburn, Mass. The scanning range of scanning head was 117 μm in x axis, 112.91 μm in y axis, and 21.9 μm in z axis. The used AFM cantilever was obtained under the trade designation PPP-NCLR-50, serial number 25298F6L376, from NANOSENSORS, Neuchâtel, Switzerland. Images were acquired in contact mode. Image size was 5 μm×5 μm. The scanning rate was 0.5 sec/line and the image resolution was 256 lines per image. The thickness of the gold layer of the nanohole array was measured to be 133 nm by comparing the height of the gold layer above the exposed polymer substrate in an area of defect in the gold layer.

### Volatile Organic Compounds (VOCs) Control Chamber

Sensor elements were exposed to the solvent at different concentrations in air. All tests were performed in air that had been passed over Drierite to remove moisture and passed over activated carbon to eliminate organic contaminates. Vapor tests were conducted using a 10 L/min dry air flow through the system. VOCs levels were generated using a syringe pump obtained from KD Scientific, Holliston, Mass. fitted with a gas tight syringe (obtained from Hamilton Company of Reno, Nev.). The syringe pump delivered the VOC indicated in Tables 2 and 3 onto a piece of filter paper suspended in a 500 mL three-necked flask. The flow of dry air passed over the paper and vaporized the solvent. Controlling the rate of delivery of VOC from the syringe pump generated concentrations of VOC indicated in Tables 2 and 3. The syringe pump was controlled by a LABVIEW program. All measurements were done at room temperature. Air concentrations of VOCs in parts per million, Cₚₚₘ, were calculated according to the following formula:

[symbol]

$$\text{?}$$

$$\text{?} \text{indicates text missing or illegible when filed}$$

where T is the experimental temperature (K), P is the experimental pressure (mmHg), q_{L} is the VOC liquid flow rate (mL/min), ρ is the density of the VOC (g/mL), q_{D} is the diluent gas flow rate (L/min), and M is the molecular weight of the VOC (g/mol). q_{D} was measured using a flow meter (model 4100, obtained from TSI of Shoreview, Minn.) and the standard air flow rate was 10 L/min at 21.11° C.

The concentration of VOC in air was verified by measurement using an IR analyzer obtained under the trade designation MIRAN from THERMO FISHER SCIENTIFIC, Waltham, Mass.

### Optoelectronic Measurement Method

A cuvette holder obtained under the trade designation CUV-FL-DA from OCEAN OPTICS was positioned sideways, as illustrate in FIG. 3. Two optical fiber probes obtained under the trade designation QP600-025-UV from OCEAN OPTICS, Largo, Fla., were each connected to opposing fiber optic connectors of the cuvette holder. One optical fiber probe was connected to a light source obtained under the trade designation HL-2000-FHSA from OCEAN OPTICS and the other optical fiber probe was connected to a spectrometer obtained under the trade designation SD2000 from OCEAN OPTICS with an analog to digital converter obtained under the trade designation ADC1000-USB from OCEAN OPTICS. A piece of nanohole array films, either coated with PIM or uncoated, as indicated in Tables 2 and 3, were placed on a cut micro cover glass (10 mm×18 mm, obtained as catalog number 48366 205 from VWR INTERNATIONAL, Radnor, Pa.). Transparent tape was used to attach the edges of the array film to the glass slide. The nanohole array film with glass slide was placed in the holder between the detector and light source. Light from the optical fiber probe connected to the light source that was transmitted by the nanohole array was collected by the optical fiber probe attached to the spectrometer. The other two optical connectors of the cuvette holder were used to pass air with concentrations of VOCs indicated in Tables 3 and 4 through the interior of the cuvette holder. The wavelength range of spectra collected was from 178.68 nm to 851.06 nm. Spectra were acquired using a computer software obtained under the trade designation SPECTRASUITE from Ocean Optics.

In case of scope mode without reference spectra, the signal sensitivity could be varied depending on detectors. Those variation can be cancelled by obtaining transmission ratio with a reference spectrum. Here, a white light source without nanohole array was used. Transmission spectra were also converted to absorbance spectra.

The obtained transmission spectra were converted to color (RGB color space) as follows. The measured reflection spectrum was constructed to International Commission on Illumination (or “CIE”) XYZ color space using color matching the CIE 1931 2° Standard Observer function. The CIE XYZ color space was linear transformed to CIE RGB space using CIE color space chromaticity coordinates (x_{R}=0.49, y_{R}=0.177. x_{G}=0.310, y_{G}=0.812, x_{B}=0.20, y_{B}=0.01). Then, Hue which is one of the main properties of a color, was computed from RGB values. Hue is defined as the degree to which a stimulus can be described as similar to or different from stimuli that are described as red, green, and blue. The color can be correlated to a location (Hue) in the color wheel from 0 degree to 360 degree. All mathematical processing was done by a customized LABVIEW program (software available from National Instruments of Austin, Tex.).

### Example 1 (EX1) and Comparative Example 1 (CE1)

Example 1 was a nanohole array with a PIM coating layer made as described below. Comparative Example 1 was a nanohole array without a PIM coating layer.

### Preparation of Example 1 (PIM Layer Coating on Nanohole Array)

The 4% wt/wt PIM solution was further diluted to 1% wt/wt in chlorobenzene (Spectrophotometric Grade, Alfa Aesar). Nanoarray film was cut in the size of 1.5 cm×1.5 cm and placed on a glass slide. 10 μL of 1% wt/wt PIM solution in chlorobenzene was applied using a micropipette on the nanoarray side. Sharply cut edges of polymer film were used to spread the solution uniformly on the entire area. The coated film was dried in oven for 1 h at 80° C. After baking, the film was kept in dry chamber under nitrogen before measurements.

After coating PIM on nanohole array, the transmitted color was close to green. The absorbance spectra were collected using optoelectronic measurement described above. The absorbance at 425.47 nm is related to the thickness of PIM layer. For calibration curve between the thickness of PIM and absorbance at 425.47 nm, PIM layer was coated on glass slides using a spin coater (Model WS 400B-8NPP/LITE spin coater, Laurell Technologies Corporation, North Wales, Pa.) at various conditions and the thickness of PIM layer was measured using a stylus profilometer (XP-1, Ambios Technology, Santa Cruz, Calif.). Absorbance at 425.47 nm was linearly proportional to the thickness of PIM layer measured from the profilometer. From the linear calibration curve, the coated thickness of PIM layer on the nanohole array is 314 nm.

### MEK Exposure to EX1

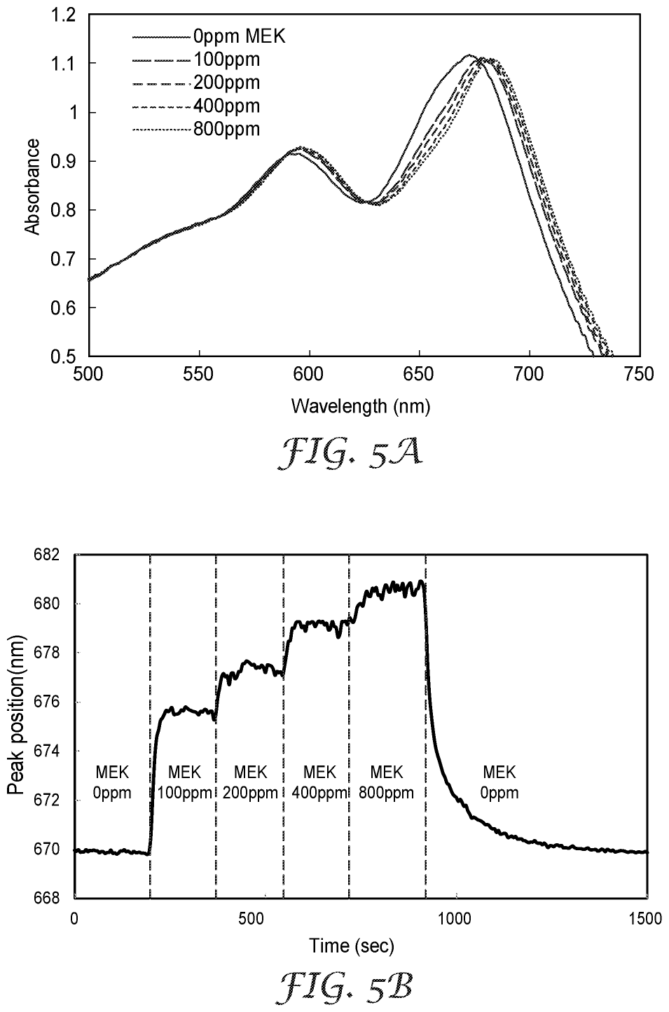

EX1 was exposed to MEK vapor concentrations of 100, 200, 400, 800 ppm. The MEK vapor was delivered to the nanohole array chamber for 3 min per each concentration. The spectra were acquired every 5 sec. FIG. 5a shows overlaid representative spectra at various MEK concentrations. The absorbance peak position around 680 nm obtained from EX1 was shifted to higher wavelength with the increase of MEK vapor concentration. Distinctive step change of absorbance peak position was observed in FIG. 5b.

Any distinctive spectral features at the various MEK concentration shown in FIG. 5 can be used to quantify MEK concentration. The distinctive spectral features include peak/valley positions, measured intensity at specified wavelengths, some mathematic processes using several intensities at various wavelengths (e.g. relative intensity ratio at two wavelengths), Hue, and so on as shown in Table 3.

### MEK Exposure to CE1

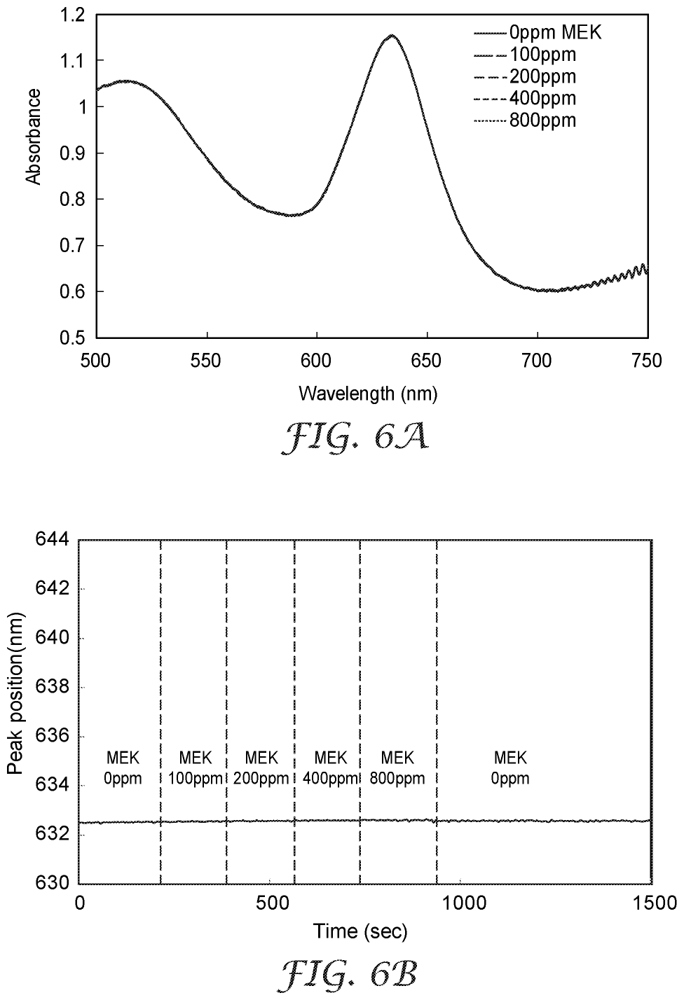

CE1 was exposed to MEK vapor at concentrations of 100, 200, 400, 800 ppm. MEK vapor was delivered to the nanohole array chamber for 3 min per each concentration. The spectra were acquired every 5 sec. FIG. 6a shows overlaid representative spectra at various MEK concentrations. All spectra at various concentrations were overlapped. No shift of absorbance peak position was observed in FIG. 6b.

Table 3 shows corresponding measurements with CE1 in comparison with Table 2.

TABLE 2

| | Abs. | Wave- | Intens. | Intens. | Intens. at | | | Trans. at | | | Abs. at | |
|---|---|---|---|---|---|---|---|---|---|---|---|---|
| | peak | length | at 660 | at 690 | 660 nm/ | Trans. | Trans. | 660 nm/ | | | 660 nm/ | |
| MEK | position | shift | nm (arb. | nm (arb. | Intens. at | at 660 | at 699 | Trans. at | Abs. at | Abs. at | Abs. at | |
| (ppm) | (nm) | (nm) | unit) | unit) | 699 nm | nm (%) | nm (%) | 699 nm | 660 nm | 669 nm | 699 nm | Hue |
| 0 | 669.89 | 0.00 | 1296 | 1609 | 0.805 | 8.60 | 14.47 | 0.594 | 1.066 | 0.839 | 1.269 | 152.4 |
| 100 | 675.63 | 5.74 | 1500 | 1386 | 1.083 | 9.97 | 12.47 | 0.799 | 1.001 | 0.904 | 1.108 | 149.4 |
| 200 | 677.41 | 7.52 | 1563 | 1324 | 1.181 | 10.39 | 11.92 | 0.872 | 0.984 | 0.924 | 1.065 | 147.5 |
| 400 | 679.29 | 9.40 | 1637 | 1263 | 1.296 | 10.88 | 11.36 | 0.957 | 0.964 | 0.945 | 1.020 | 146.3 |
| 800 | 680.63 | 10.74 | 1690 | 1222 | 1.382 | 11.23 | 11.00 | 1.021 | 0.950 | 0.959 | 0.991 | 145.0 |

TABLE 3

| | Abs. | Wave- | Intens. | Intens. | Intens. at | | | Trans. at | | | Abs. at | |
|---|---|---|---|---|---|---|---|---|---|---|---|---|
| | peak | length | at 660 | at 690 | 660 nm/ | Trans. | Trans. | 660 nm/ | | | 660 nm/ | |
| MEK | position | shift | nm (arb. | nm (arb. | Intens. at | at 660 | at 699 | Trans. at | Abs. at | Abs. at | Abs. at | |
| (ppm) | (nm) | (nm) | unit) | unit) | 699 nm | nm (%) | nm (%) | 699 nm | 660 nm | 669 nm | 699 nm | Hue |
| 0 | 632.52 | 0.00 | 2371 | 2762 | 0.858 | 15.75 | 24.87 | 0.633 | 0.803 | 0.604 | 1.328 | 37.2 |
| 100 | 632.55 | 0.03 | 2382 | 2769 | 0.860 | 15.83 | 24.94 | 0.635 | 0.800 | 0.603 | 1.327 | 37.2 |
| 200 | 632.56 | 0.05 | 2380 | 2767 | 0.860 | 15.81 | 24.92 | 0.635 | 0.801 | 0.604 | 1.327 | 37.2 |
| 400 | 632.61 | 0.09 | 2378 | 2768 | 0.859 | 15.80 | 24.92 | 0.634 | 0.801 | 0.603 | 1.328 | 37.2 |
| 800 | 632.59 | 0.07 | 2378 | 2775 | 0.857 | 15.81 | 24.99 | 0.632 | 0.801 | 0.602 | 1.330 | 37.2 |

Thus, the present disclosure provides, among other things, sensor elements and sensors comprising the sensor elements. Various features and advantages of the present disclosure are set forth in the following claims.

1. A sensor element comprising, in order:
an absorptive layer having first and second major surfaces, the absorptive layer comprising a polymer of intrinsic microporosity having an average pore volume of at least 0.1 nm³;
a metallic layer disposed on the second major surface of the absorptive layer, the metallic layer having first and second opposed major surfaces and a plurality of openings each extending from the first to the second major surface of the metallic layer, the openings having a pitch in a range from 50 nm to 5000 nm, wherein the openings have an opening size in a range from 5% to 95% percent of the pitch; and
an optically transparent, dielectric substrate having first and second opposed major surfaces with the metallic layer disposed on the first major surface of the substrate.

2. The sensor element of claim 1, wherein the openings are one of circular, oval, or polygonal (e.g., triangular, square, or rectangular).

3. The sensor element of claim 1, wherein the polymer comprises a homopolymer having a monomeric unit selected from the group consisting of

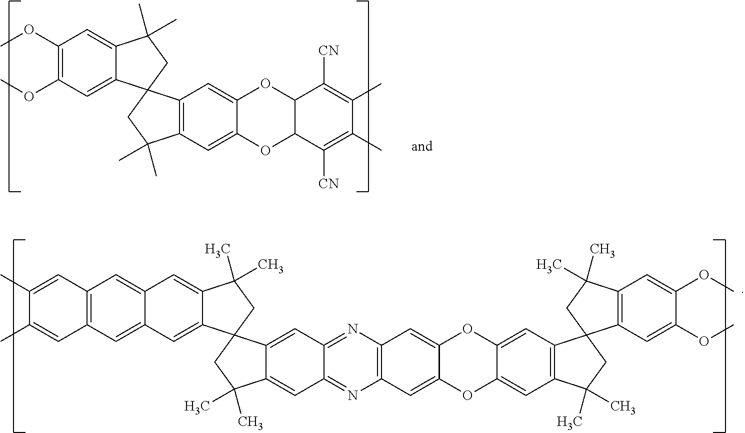

4. The sensor element of claim 1, wherein the absorptive layer has an average thickness in a range from 10 nm to 10,000 nm.

5. The sensor element of claim 1, wherein the metallic layer comprises at least one of gold, silver, copper, platinum, ruthenium, nickel, palladium, rhodium, iridium, chromium, aluminum, iron, lead, tin, zinc, a combination thereof, and an alloy thereof.

6. The sensor element of claim 1, wherein the metallic layer has an average thickness in a range from 5 nm to 1000 nm.

7. The sensor element of claim 1, wherein the openings have a square lattice, rectangular lattice, hexagonal lattice, rhombic lattice, or parallelogrammic lattice.

8. The sensor element of claim 1, wherein the substrate comprises at least one of an optical transparent dielectric inorganic layer and a polymeric layer.

9. The sensor element of claim 1, wherein the substrate comprises an optical transparent dielectric inorganic layer comprises at least one of glass, SiN and SiO₂.

10. The sensor element of claim 1, wherein the substrate comprises a polymeric layer comprising at least one of polyethylene terephthalate, poly(methyl methacrylate), polyvinyl chloride, polyethylene, polypropylene, styrene methyl methacrylate, polycarbonate, polystyrene and copolymers thereof.

11. The sensor element of claim 1 in the form of a roll.

12. A sensor for organic solvent comprising the sensor element of claim 1.

13. A sensor comprising:
a light source;
the sensor element of claim 1; and
a detector that measures optical transmission through the sensor element.

14. The sensor of claim 13, wherein the light source is a single wavelength light source.

15. The sensor of claim 13, wherein the light source is a multiple wavelengths light source.

16. The sensor of claim 13, wherein the light source emits (a) at least one wavelength of ultraviolet (UV) light, (b) at least one wavelength of visible light, or (c) at least one wavelength of infrared (IR) light.

17. The sensor according to claim 13, wherein the light source comprises a laser.

18. The sensor of claim 1, wherein the detector comprises at least one photodetector.
